(19) United States
(12) Patent Application Publication
(10) Pub. No.: US 2009/0076120 A1
(43) Pub. Date: Mar. 19, 2009

(54) Crystal of Aminopyrrolidine Derivative and Production Method Thereof

(75) Inventors: Takumi Takeyasu, Yamaguchi (JP); Yoshinori Sato, Yamaguchi (JP); Asahi Kawana, Yamaguchi (JP); Yuji Takahashi, Tokyo (JP); Yuji Ishikawa, Kanagawa (JP); Kaoru Suda, Fukushima (JP)

Correspondence Address: SUGHRUE MION, PLLC, 2100 PENNSYLVANIA AVENUE, N.W., SUITE 800, WASHINGTON, DC, 20037, US

(73) Assignee: TEIJIN PHARMA LIMITED, Chiyoda-ku, Tokyo` (JP)

(21) Appl. No.: 11/887,888

(22) PCT Filed: Apr. 6, 2006
(86) PCT No.: PCT/JP2006/307784

§ 371 Date: Nov. 13, 2007

(30) Foreign Application Priority Data
Apr. 7, 2005 (JP) 2005-110854

(51) Int. Cl. A61K 31/404; C07D 403/02; A61P 29/00; A61P 11/00; A61P 9/00

(52) U.S. Cl. 514/414; 548/465

(57) ABSTRACT

Two crystal forms of (R)-3-[2-(2-amino-5-trifluoromethoxybenzamido)acetamido]-1-(6-methylindol-3-ylmethyl)pyrrolidine which exhibit specific X-ray powder diffraction patterns or infrared absorption spectra, amorphous form thereof, a pharmaceutical composition containing the crystal or amorphous form as an active ingredient, as well as methods for preparing them are provided.

### FIELD OF THE INVENTION

The present invention relates to crystal forms of (R)-3-[2-(2-amino-5-trifluoromethoxybenzamido)acetamido]-1-(6-methylindol-3-ylmethyl)pyrrolidine, a production method thereof, an amorphous form thereof and a pharmaceutical composition containing thereof. The compound has a chemokine receptor antagonistic activity in the living body and can be used as a preventive and therapeutic agent for inflammatory disease, allergic disease, respiratory disease or cardiovascular disease.

### BACKGROUND ART

When a compound forms two or more types of crystals, these different crystalline forms are called polymorphs. It is generally known that the stability is varied depending on each crystalline form (crystal form) of the polymorph. For example, it has been described in Japanese Published Unexamined Application No. 62-226980 that two types of crystalline forms of prazosin hydrochloride differ in the stability, affecting the results of the long term storage stability. Further, it has been described in Japanese Published Unexamined No. 64-71816 that a specific crystalline form among different crystalline forms of buspirone hydrochloride is advantageous in terms of the maintenance of particular physical properties under the conditions of storage and production.

In general, in the production of drug substance, it is advantageous to obtain drug substance in a crystalline form regarding the storage stability and the control of production process and the like of drug substance and a pharmaceutical composition.

Further, when a compound which exists in two or more crystalline forms is used as a pharmaceutical product, physical and chemical behaviors such as melting point, solubility or stability or the like, and pharmacokinetics (absorption, distribution, metabolism, excretion or the like) are varied depending on each crystal, resulting in different biological properties such as pharmacological effect. In order to assure the consistency of these properties as a pharmaceutical product, it is often required to produce a drug substance of a specific crystal form. Moreover, in the production process of drug substance, it often becomes important to precipitate a specific crystalline form in the crystallization operation in order to maintain the yield and purification effect constant.

Since it is impossible to predict the presence of a crystalline polymorph from a structure of a compound, it is considered to be important to find out a crystalline form(s) in the development of a pharmaceutical product.

As described in International Publication WO 99/25686, (R)-3-[2-(2-amino-5-trifluoromethoxybenzamido)acetamido]-1-(6-methylindol-3-ylmethyl)pyrrolidine has been known to have a chemokine receptor antagonistic activity. However, no description is made for the crystal or the crystalline polymorph of the compound in the above literature.

Incidentally, a chemokine such as MCP-1 is a proteinic factor having an migration-inducing and activating activities and the like of leukocyte, which is a group of inflammatory and immuno-modifying polypeptide produced at an inflammatory site by various cells such as macrophages, monocytes, eosinophils, neutrophils, fibroblasts, endothelial cells, smooth muscle cells and mast cells. The tissue infiltration of a blood leukocyte component such as monocytes and lymphocytes plays a critical role in the progress and maintenance of diseases described below. That is, they include atherosclerosis, rheumatoid arthritis, psoriasis, asthma, ulcerative colitis, nephritis (nephropathy), multiple sclerosis, pulmonary fibrosis, cardiomyopathy, hepatitis, pancreatitis, sarcoidosis, Crohn's disease, endometriosis, congestive heart failure, viral meningitis, cerebral infarction, neuropathy, Kawasaki disease, diabetes, sepsis and the like.

### DISCLOSURE OF THE INVENTION

An object of the present invention is to provide a crystal of (R)-3-[2-(2-amino-5-trifluoromethoxybenzamido)acetamido]-1-(6-methylindol-3-ylmethyl)pyrrolidine, a method of production thereof and an amorphous form thereof.

Further, another object of the present invention is to provide a preventive and therapeutic agent for inflammatory disease, allergic disease, respiratory disease or cardiovascular disease which have a chemokine receptor antagonistic activity.

As a result of an intensive study, the inventors have found that a crystal of (R)-3-[2-(2-amino-5-trifluoromethoxybenzamido)acetamido]-1-(6-methylindol-3-ylmethyl)pyrrolidine exists in two types of crystalline forms as well as in an amorphous form. Moreover, the inventors have found that one of the crystalline forms is preferable as a drug substance of a pharmaceutical composition or as a production intermediate thereof and have achieved the present invention.

That is, the present invention is a crystal of (R)-3-[2-(2-amino-5-trifluoromethoxybenzamido)acetamido]-1-(6-methylindol-3-ylmethyl)pyrrolidine.

Further, the present invention is a crystal of (R)-3-[2-(2-amino-5-trifluoromethoxybenzamido)acetamido]-1-(6-methylindol-3-ylmethyl)pyrrolidine (crystal A) exhibiting an X-ray powder diffraction pattern having characteristic peaks expressed in the reflection angle 2θ (degree) at about 5.7, 8.4, 15.2, 16.9, 19.7, 20.9, 21.3, 21.7 and 24.1. That is, the crystal exhibits the X-ray powder diffraction pattern which is approximately shown in FIG. 1.

Furthermore, the crystal has an infrared absorption spectrum in potassium bromide having absorption peaks expressed in the wavenumber (cm⁻¹) at approximately 1651, 1637, 1583, 1556, 1294, 1265, 1223, 1205, 1169, 1155, 1097, 1051, 1007, 966, 885, 835 and 804. Namely, the crystal exhibits an infrared absorption spectrum in potassium bromide which is approximately shown in FIG. 3.

The present invention is a crystal of (R)-3-[2-(2-amino-5-trifluoromethoxybenzamido)acetamido]-1-(6-methylindol-3-ylmethyl)pyrrolidine (crystal B) exhibiting an X-ray powder diffraction pattern having characteristic peaks expressed in the reflection angle 2θ (degree) at about 9.6, 11.3, 15.5, 16.3, 16.3, 19.3, 20.0, 20.5, 20.9, 22.7, 23.3, 24.2, 27.2, 27.8 and 31.6. That is, the crystal exhibits the X-ray powder diffraction pattern which is approximately shown in FIG. 2.

Furthermore, the crystal has an infrared absorption spectrum in potassium bromide having absorption peaks expressed in the wavenumber (cm⁻¹) at approximately 1639, 1556, 1265, 1223, 1167, 1149, 1119, 1099, 1055, 1011, 960, 891, 858, 825 and 796. Namely, the crystal exhibits an infrared absorption spectrum in potassium bromide which is approximately shown in FIG. 4.

Incidentally, the expressions such as “at about 5.7 . . . [snip] . . . 24.1” in the above-mentioned X-ray powder diffraction data and “at approximately 1651 . . . [snip] . . . 804” in above-mentioned infrared spectrum data are used in a sense that a variation within measurement accuracy and error is acceptable to the extent that the identity of a crystalline form may be confirmed in view of technical common knowledge. Therefore, the figure ranges are not made unclear by these expressions.

The present invention further provides a method of producing these crystals. Examples of the method include;

a method of producing crystal A by cooling crystallization from a solution of (R)-3-[2-(2-amino-5-trifluoromethoxybenzamido)acetamido]-1-(6-methylindol-3-ylmethyl)pyrrolidine in methanol, ethanol, 2-propanol, ethyl acetate, n-propyl acetate, tetrahydrofuran, 2-butanone, acetonitrile, toluene, hexane, heptane, water or in a mixed solvent of two kinds or more selected thereof;
a method of producing crystal A by anti-solvent crystallization, wherein toluene, hexane, heptane, water or a mixed solvent of two kinds or more selected thereof is added to a solution of (R)-3-[2-(2-amino-5-trifluoromethoxybenzamido)acetamido]-1-(6-methylindol-3-ylmethyl)pyrrolidine in methanol, ethanol, 2-propanol, ethyl acetate, n-propyl acetate, tetrahydrofuran, 2-butanone, acetonitrile, toluene, hexane, heptane, water or a mixed solvent of two kinds or more selected thereof; a method of producing the crystal A by carrying out a cooling crystallization step from a solution of (R)-3-[2-(2-amino-5-trifluoromethoxybenzamido)acetamido]-1-(6-methylindol-3-ylmethyl)pyrrolidine in methanol, ethanol, 2-propanol, ethyl acetate, n-propyl acetate, tetrahydrofuran, 2-butanone, acetonitrile, toluene, hexane, heptane, water or in a mixed solvent of two kinds or more selected thereof, simultaneously with or before an anti-solvent crystallization step, wherein toluene, hexane, heptane, water or a mixed solvent of two kinds or more selected thereof is added further (the cooling crystallization step and the anti-solvent crystallization step may be carried out in this order, or the latter may be carried out simultaneously with the former or during the former, further the both steps may be finished simultaneously or either may finished earlier than the other);
a method of producing the crystal A by suspending (R)-3-[2-(2-amino-5-trifluoromethoxybenzamido)acetamido]-1-(6-methylindol-3-ylmethyl)pyrrolidine in methanol, ethanol, 2-propanol, ethyl acetate, n-propyl acetate, tetrahydrofuran, 2-butanone, acetonitrile, toluene, hexane, heptane, water or a mixed solvent of two kinds or more selected thereof;
a method of producing crystal A by the neutralizing crystallization by adding an alkaline solution or a water soluble organic solvent containing the alkaline solution to a solution of an acid salt or mixture of acid salts of (R)-3-[2-(2-amino-5-trifluoromethoxybenzamido)acetamido]-1-(6-methylindol-3-ylmethyl)pyrrolidine in water, or in a mixed solvent of water and one or more solvent selected from water soluble organic solvents;
a method of producing the crystal B by the neutralizing crystallization by adding an acid salt of (R)-3-[2-(2-amino-5-trifluoromethoxybenzamido)acetamido]-1-(6-methylindol-3-ylmethyl)pyrrolidine in water or in a mixed solvent of water and one or more solvents selected from water soluble organic solvents to an alkaline solution or a water soluble organic solvent containing the alkaline solution.

The present invention is further an amorphous form of (R)-3-[2-(2-amino-5-trifluoromethoxybenzamido)acetamido]-1-(6-methylindol-3-ylmethyl)pyrrolidine.

The present invention is further a pharmaceutical composition containing any of the above-mentioned crystals or amorphous form, or a mixture of the crystal or the amorphous form selected therefrom as an active ingredient.

The present invention is further a composition having a chemokine receptor antagonistic activity containing, as an active ingredient, any of the above-mentioned crystals or amorphous form, or a mixture of the crystal or the amorphous form selected therefrom.

The present invention is further a preventive drug or a therapeutic drug for inflammatory disease, allergic disease, respiratory disease or cardiovascular disease containing, as an active ingredient, any of the above-mentioned crystals or amorphous form, or a mixture of the crystal or the amorphous form selected therefrom.

According to the present invention, there may be provided two kinds of crystal of (R)-3-[2-(2-amino-5-trifluoromethoxybenzamido)acetamido]-1-(6-methylindol-3-ylmethyl)pyrrolidine, a method of production thereof and an amorphous form thereof. These crystals has a chemokine receptor antagonistic activity and are used as an active ingredient of a preventive drug or a therapeutic drug for inflammatory disease, allergic disease, respiratory disease or cardiovascular disease.

### BRIEF DESCRIPTION OF THE DRAWINGS

FIG. 1 shows an XRD diagram of crystal A of the present invention.

FIG. 2 shows an XRD diagram of crystal B of the present invention.

FIG. 3 shows an IR diagram of crystal A of the present invention.

FIG. 4 shows an IR diagram of crystal B of the present invention.

### BEST MODE FOR CARRYING OUT THE INVENTION

The crystals of the present invention are characterized by an X-ray powder diffraction pattern and/or an infrared absorption peaks in potassium bromide. These crystals exhibit a characteristic X-ray powder diffraction pattern (XRD), each of which has characteristic 2θ values. In addition, these crystals each exhibit a characteristic absorption pattern in an infrared spectrophotometry (IR).

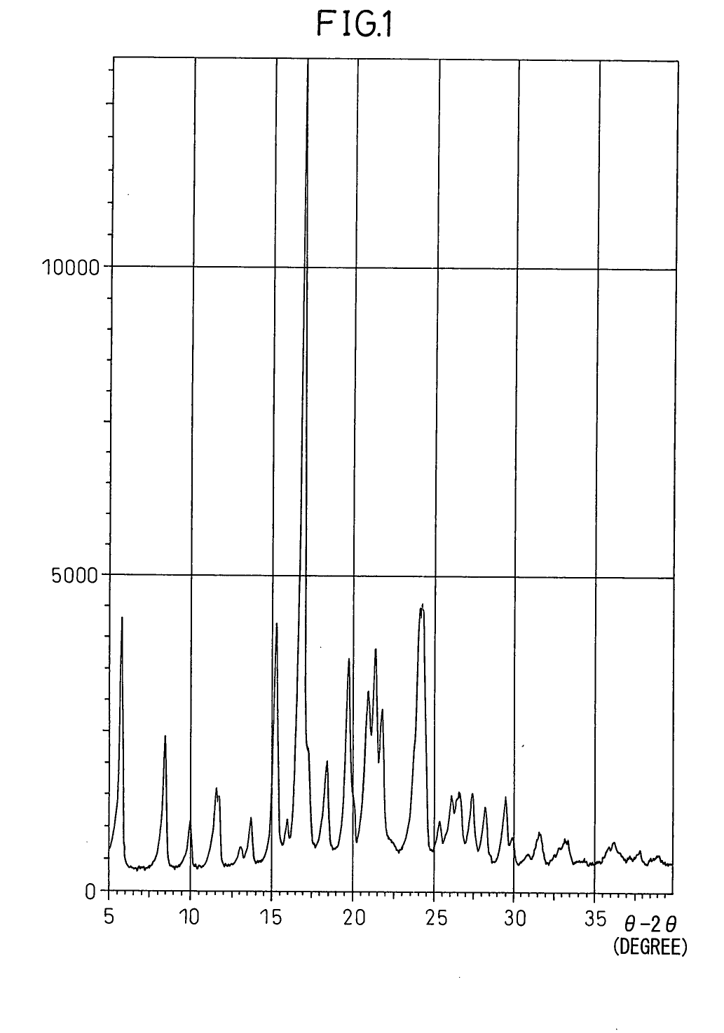

Crystal A of the present invention has an X-ray powder diffraction pattern having peaks expressed in reflection angle 2θ (degree) at about 5.7, 8.4, 15.2, 16.9, 19.7, 20.9, 21.3, 21.7 and 24.1. More particularly, the crystal exhibits an X-ray powder diffraction pattern having characteristic peaks shown in table 1 (Refer to FIG. 1). In the X-ray powder diffraction pattern intensity in table 1, Iₘₐₓ represents the peak intensity with the highest intensity of each crystal and I represents the intensity of each peak. Further, a 2θ values of an X-ray powder diffraction pattern may be varied by a range of 0.5 degrees depending on the sample state and measuring conditions. In the X-ray powder diffraction, due to the nature of the data, the overall pattern is important in verifying the identity of a crystal. The relative intensity should not be strictly interpreted because it may be varied to some extent depending on the crystal growth direction, particle sizes and measurement conditions.

TABLE 1
(Crystal A)

| Diffraction Angle (2θ, °) | Intensity (I/Iₘₐₓ × 100) |
|---|---|
| 5.7 | 18 |
| 8.4 | 16 |
| 15.2 | 17 |
| 16.9 | 100 |
| 19.7 | 28 |
| 20.9 | 21 |
| 21.3 | 27 |
| 21.7 | 16 |
| 24.1 | 42 |

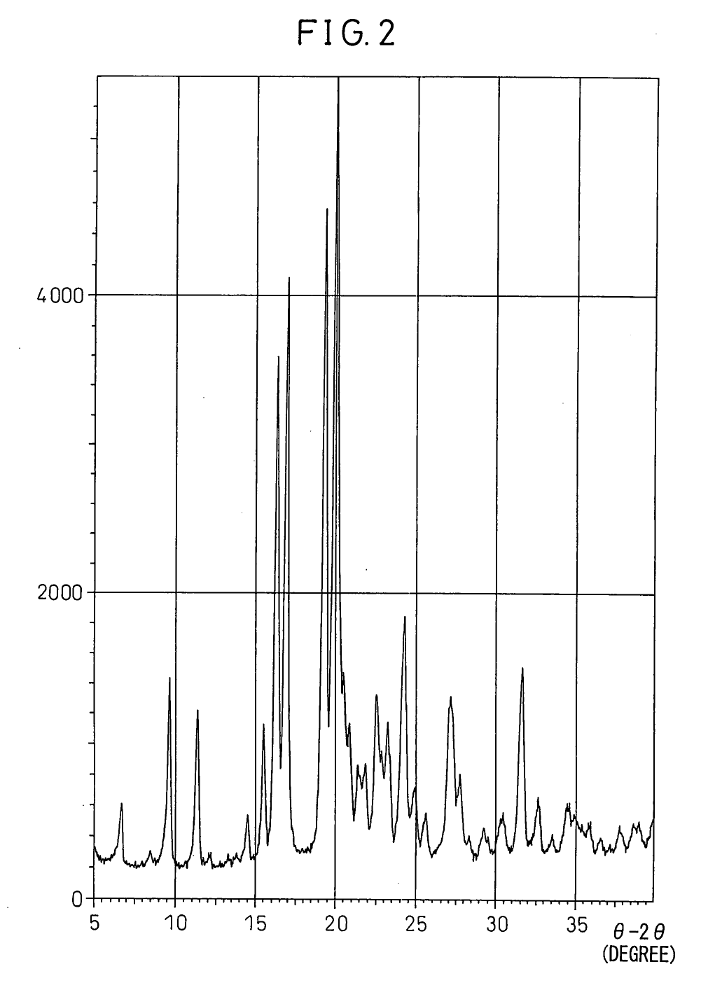

Crystal B of the present invention has an X-ray powder diffraction pattern peaks expressed in the reflection angle 2θ (degree) at about 9.6, 11.3, 15.5, 16.3, 16.9, 19.3, 20.0, 20.5, 22.7, 24.2, 27.2 and 31.6, and more particularly exhibits an X-ray powder diffraction pattern having characteristic peaks shown in Table 2 (Refer to FIG. 2).

TABLE 2
(Crystal B)

| Diffraction Angle (2θ, °) | Intensity (I/Iₘₐₓ × 100) |
|---|---|
| 9.6 | 24 |
| 11.3 | 19 |
| 15.5 | 16 |
| 16.3 | 65 |
| 16.9 | 76 |
| 19.3 | 85 |
| 20.0 | 100 |
| 20.5 | 21 |
| 22.7 | 18 |
| 24.2 | 30 |
| 27.2 | 20 |
| 31.6 | 25 |

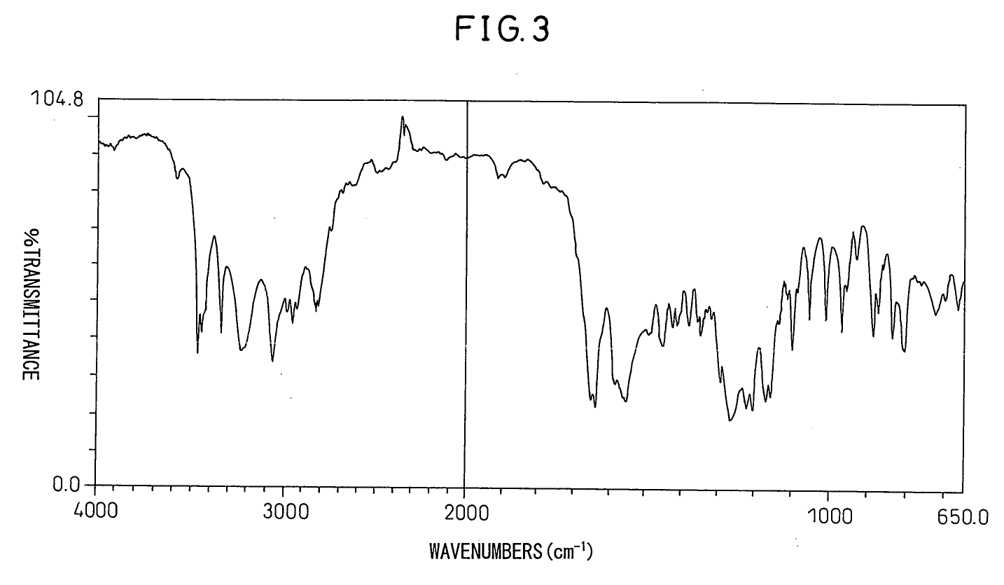

Crystal A has peaks expressed in wavenumber (cm⁻¹) at approximately 1651, 1637, 1583, 1556, 1294, 1265, 1223, 1205, 1169, 1155, 1097, 1051, 1007, 966, 885, 835 and 804, according to infrared spectrophotometry (Refer to FIG. 3).

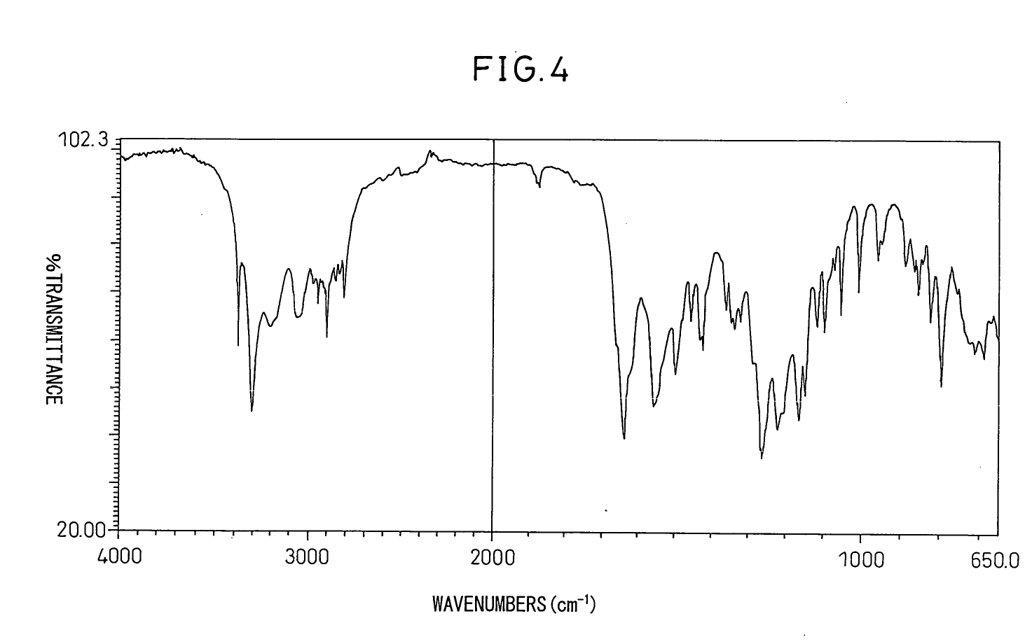

Crystal B of the present invention has peaks expressed in wavenumber (cm⁻¹) at approximately 1639, 1556, 1265, 1223, 1167, 1149, 1119, 1099, 1055, 1011, 960, 891, 858, 825 and 796 (Refer to FIG. 4).

Further, the wave number observed by the infrared spectrophotometry of the present invention may be varied by 5 cm⁻¹ depending on the measurement conditions and the sample state and the like.

The crystals of the present invention each may be obtained by the various production methods mentioned above, typical examples of which will be described below.

Incidentally, the compound of the present invention, (R)-3-[2-(2-amino-5-trifluoromethoxybenzamido)acetamido]-1-(6-methylindol-3-ylmethyl)pyrrolidine, can be synthesized by a method described in International Publication WO 99/25686 and the like. For example, a t-butoxycarbonyl group is removed from (R)-2-(t-butoxycarbonylamino)-N-[1-(6-methylindol-3-ylmethyl)pyrrolidin-3-yl]acetamide under acidic conditions to obtain a 2-aminoacetamide derivative, followed by condensing 5-trifluoromethoxyanthranilic acid using a condensing agent such as 1-ethyl-3-(3-dimethylaminopropyl)carbodiimide hydrochloride to obtain (R)-3-[2-(2-amino-5-trifluoromethoxybenzamido)acetamido]-1-(6-methylindol-3-ylmethyl)pyrrolidine.

In the two types of crystals, the stability of crystal A is higher than that of crystal B.

Crystal A can be crystallized by a cooling crystallization process from a solution in various solvents, by a suspension process in which it is suspended in various solvents, by an anti-solvent crystallization process in which poor solvent is added to a solution, or by a neutralizing crystallization process in which an alkaline solution or a water soluble organic solvent containing the alkaline solution is added, preferably dropwise, to a solution of an acid salt in water or in a mixed solvent of water and a water soluble organic solvent. Examples of the solvent include acetone, ethanol, isobutyl acetate, isopropyl acetate, ethyl acetate, butyl acetate, propyl acetate, methyl acetate, diethyl ether, t-butyl methyl ether, 1-butanol, 2-butanol, 1-propanol, 2-propanol, heptane, 1-pentanol, 4-methylpentanone, 2-butanone, 3-methyl-1-butanol, 2-methyl-1-propanol, tetrahydrofuran, acetonitrile, cyclohexane, 1,2-dimethoxyethane, 1,4-dioxane, 2-ethoxyethanol, hexane, pentane, methanol, 2-ethoxymethanol, methylcyclohexane, tetralin, toluene, xylene, water or a mixed solvent of two kinds or more selected thereof.

As a more preferred solvent from the economic and industrial point of view, there may be mentioned methyl acetate, ethyl acetate, propyl acetate, isopropyl acetate, butyl acetate, 4-methyl-2-pentanone, 2-butanone, acetone, tetrahydrofuran, acetonitrile, hexane, cyclohexane, heptane, toluene, xylene, methanol, ethanol, 1-propanol, 2-propanol, water or a mixed solvent of two kinds or more selected thereof. More preferably, there may be mentioned tetrahydrofuran, ethanol, 2-propanol, 2-butanone, ethyl acetate, isopropyl acetate, hexane, heptane, toluene, water or a mixed solvent of two kinds or more selected thereof.

In the case where crystal A is to be obtained by a cooling crystallization process, a suspension process, an anti-solvent process and a neutralizing crystallization process, a temperature of a solution is not specifically limited but it is preferably lower than the boiling point of the solvent used. The solvent amount is not specifically limited but it is preferably a 5- to 100-fold amount, more preferably a 50-fold amount or less and most preferably a 20-fold amount or less. Here, 1-fold amount means a 1 mL of solvent to 1 g of raw material.

Further, in the case where crystal A is obtained by these crystallization processes, it is effective to add seed crystals with the same crystalline form as that of the crystal of interest. The amount is typically in the range of about 0.01 to 20% of the raw material, preferably in the range of 0.1 to 10% of the raw material. The solution temperature at addition is required to be within the supersaturation range of the crystal to be obtained.

Crystal B may be obtained by the neutralizing crystallization process in which a solution of an acid salt of (R)-3-[2-(2-amino-5-trifluoromethoxybenzamido)acetamido]-1-(6-methylindol-3-ylmethyl)pyrrolidine in water or in a mixed solvent of water soluble organic solvent containing water is added dropwise to an alkaline solution or a water soluble organic solvent containing the alkaline solution.

Examples of the solvent include acetone, ethanol, 1-propanol, 2-propanol, tetrahydrofuran, 1,4-dioxane, 2-ethoxyethanol, methanol, 2-ethoxymethanol, water or a mixed solvent of two kinds or more selected thereof.

As a more preferable solvent from the economic and industrial point of view, there may be mentioned acetone, tetrahydrofuran, methanol, ethanol, 1-propanol, 2-propanol, water or a mixed solvent of two or more kinds selected thereof. More preferably, there may be mentioned methanol, ethanol, 2-propanol, water or a mixed solvent of two kinds or more selected thereof.

In the case where crystal B is obtained by the neutralizing crystallization process, a temperature of the solution is not specifically limited but it is preferably lower than the boiling point of the solvent used, more preferably room temperature which is 30° C. or less. The solvent amount is not specifically limited but it is preferably a 5- to 100-fold amount, more preferably a 50-fold amount or less and most preferably a 20-fold amount or less. Here, 1-fold amount means 1 mL of solvent to 1 g of raw material.

In the present invention, “an acid salt” is referred to as a salt of an inorganic acid such as hydrochloric acid, hydrobromic acid, sulfuric acid, phosphoric acid, carbonic acid and the like or an organic acid such as maleic acid, citric acid, malic acid, tartaric acid, fumaric acid, methanesulfonic acid, trifluoroacetic acid, formic acid and the like, and there may be preferably mentioned a hydrochloride.

Further, in the present invention, “an alkaline solution” is referred to as a basic aqueous solution of a metal hydroxide such as an alkaline metal or alkaline earth metal, an alkaline metal carbonate, ammonia or an organic amine and other alkalis, and there may be preferably mentioned a solution of the alkaline metal hydroxide such as a sodium hydroxide solution and a potassium hydroxide solution.

Crystal B or a crystalline mixture containing crystal B may be transformed to crystal A under the following conditions.

As a solvent used in the transition, there may be used methanol, ethanol, 2-propanol, ethyl acetate, n-propyl acetate, tetrahydrofuran, 2-butanone, acetonitrile, toluene, hexane, heptane, water or a mixed solvent of two kinds or more selected thereof. The solvent amount in the transition is required to be set such that a suspension state is maintained at a temperature in the transition, and is usually a 2- to 100-fold amount of the crystal to be transformed, preferably a 50-fold amount or less and more preferably a 20-fold amount or less.

Any of the crystallization processes described above is effective as a method for obtaining individual crystalline form. However, since there is a tendency of increase in the specific decomposed substance when a solution of (R)-3-[2-(2-amino-5-trifluoromethoxybenzamido)acetamido]-1-(6-methylindol-3-ylmethyl)pyrrolidine is heated, there may be mentioned the suspension process, the anti-solvent process or the neutralizing crystallization process, in which no heating is needed as a preferable crystallization process from the viewpoint of the pharmaceutical production. Among them, the neutralizing crystallization process is more preferable.

When a mixture of two types of crystals is to be obtained, the mixture can be produced at a time in addition to producing and mixing each crystal. However, in order to obtain a mixture with a desired mixing ratio, setting of conditions is required to be made based on a detailed preliminary study.

For the quantification of the mixing ratio, the ratio may be calculated by an analysis method such as an X-ray powder diffraction pattern, an infrared absorption spectrum, a thermal analysis and the like, although it depends on a combination or a ratio of crystals. In such a case, for example, a solvent mediated transition is preferable in the point that the mixing ratio may be sequentially monitored.

Although each crystal of the present invention may be distinguishable from other crystalline forms by a characteristic X-ray powder diffraction pattern and/or an infrared absorption spectrum, contamination from other crystalline forms cannot be completely ruled out. In a case where a specific crystalline form is solely obtained, the contamination may be acceptable to a degree that the contamination is not detected at least by an X-ray powder diffraction pattern and an infrared absorption spectrum. In practice, even when each specific crystalline form is used as a drug substance, a minimal amount of contamination from a small amount of other crystalline forms may be permitted.

Any of the two types of crystals of the present invention or the mixture thereof may be used as an active ingredient of pharmaceutical compositions.

A crystal of (R)-3-[2-(2-amino-5-trifluoromethoxybenzamido)acetamido]-1-(6-methylindol-3-ylmethyl)pyrrolidine of the present invention is excellent compared to non-crystalline forms with respect to handling, reproducibility and stability in production, storage stability and the like.

Crystal A is preferably used as a stable crystal which is excellent with respect to reproducibility and stability in production and storage stability. Further, crystal B is also useful as a starting material (production intermediate) for transition to crystal A because it is a crystal and thus easily handled.

A preparation containing a crystal or an amorphous form of (R)-3-[2-(2-amino-5-trifluoromethoxybenzamido)acetamido]-1-(6-methylindol-3-ylmethyl)pyrrolidine of the present invention may be prepared by using a carrier, an excipient and other additives which are typically used for pharmaceutical preparations. As the carrier and the excipient for formulation which may be used in the form of solid or liquid, there may be mentioned lactose, magnesium stearate, starch, talc, gelatin, agar, pectin, gum arabic, olive oil, sesame oil, cacao butter, ethylene glycol and others in common use. Administration may be made orally in the form of tablets, pills, capsules, granules, powers, solutions and the like or parenterally in the form of solutions for intravenous injection, intramuscular injection and the like, suppositories, percutaneous administration and the like.

A dose of a crystal or an amorphous form of (R)-3-[2-(2-amino-5-trifluoromethoxybenzamido)acetamido]-1-(6-methylindol-3-ylmethyl)pyrrolidine of the present invention, which differs depending on the kind of disease, route of administration, and symptoms, age, sex and weight of patients and the like, is generally about 1 to 500 mg/day/person, preferably 10 to 300 mg/day/person for oral administration. The dose is about 0.1 to 100 mg/day/person, preferably 0.3 to 30 mg for parenteral administration such as intravenous, subcutaneous, intramuscular, percutaneous, rectal, intranasal, ophthalmic or inhalation administration and the like.

Further, when a crystal or an amorphous form of (R)-3-[2-(2-amino-5-trifluoromethoxybenzamido)acetamido]-1-(6-methylindol-3-ylmethyl)pyrrolidine of the present invention is used as a preventive drug, it can be administered in advance depending on each symptom in accordance with a well-known method.

The diseases targeted by the preventive drug or the therapeutic drug of the present invention include atherosclerosis, rheumatoid arthritis, psoriasis, asthma, ulcerative colitis, nephritis (nephropathy), multiple sclerosis, pulmonary fibrosis, cardiomyopathy, pancreatitis, sarcoidosis, Crohn's disease, endometriosis, congestive heart failure, viral meningitis, cerebral infarction, neuropathy, Kawasaki disease, diabetes and sepsis and the like.

### EXAMPLES

Hereinafter, the methods of obtaining the crystal of the present invention will be explained by examples.

However, the present invention is not limited by these examples.

The analysis of the crystals of the present invention was performed under the following conditions.

Measurement Conditions for an X-Ray Powder Diffraction Pattern

Equipment: RIGAKU ROTAFLEX RU300 (X-ray powder diffraction pattern measurement equipment)
X-ray source: Cu-Kα (λ=1.5418×10⁻¹⁰ m), 50 kV-200 mA
Slit: DS1°-SS1°-RS 0.15 mm-graphite monochrometer-0.45 mm
Method: 2θ-θ scan, 0.05 step/1 sec, scan range 5 to 40°

### Or,

### Equipment: Shimadzu XRD-6000

X-ray source: Cu-Kα (λ=1.5418×10⁻¹⁰ m), 40 kV-40 mA
Slit: DS1°-SS1°-RS 0.15 mm-graphite monochrometer-0.45 mm
Method: 2θ-θ scan, 0.02 step/1 sec, scan range 5 to 400

Measurement Conditions for an Infrared Absorption Spectrum

### Equipment: HORIBA FT-270 or Shimadzu FT-IR 8600

In accordance with the potassium bromide method, measurements were made by FT-IR (Resolution: 4, SCAN: 40, Gain: AUTO).

Differential Scanning Calorimetry (DSC) Conditions

### Equipment: Shimadzu

### Differential Scanning Calorimeter: DSC-50

### Thermal Analysis System: TA-50

Reference: empty

### Scan Speed: 10° C./min

### Sampling: 0.5 sec.

### Upper Limit: 230° C.

### Lower Limit: 30° C.

### Atmosphere: Nitrogen

### Sampling Pan: Aluminum (Sealed)

### Sample Weight: 1 to 3 mg

Incidentally, although each crystal of the present invention may be identified by DSC, the values of DSC may be varied depending on the measurement conditions and sample conditions. Therefore, the DSC values shown in examples cannot be identified as absolute values.

### Examples 1 to 16

### Production of Crystal A of (R)-3-[2-(2-amino-5-trifluoromethoxybenzamido)acetamido]-1-(6-methylindol-3-ylmethyl)pyrrolidine

The results of the crystallization conditions for obtaining crystal A are summarized in Table 3.

TABLE 3

| | Solvent | | | | |
|---|---|---|---|---|---|
| Example | crystallization | Temperature | Yield | | DSC |
| No. | process | (° C.) | (%) | Identification | (° C.) |
| 1 | EtOH | 70 | 48 | XRD | — |
| | Cooling | | | | |
| | crystallization | | | | |
| 2 | CH₃CN | 90 | 67 | XRD | — |
| | Cooling | | | | |
| | crystallization | | | | |
| 3 | EtOH/Hexane | 80 | 47 | XRD | — |
| | Cooling | | | | |
| | crystallization | | | | |
| 4 | EtOH/Water | 80 | 71 | XRD, IR | — |
| | Cooling | | | | |
| | crystallization | | | | |
| 5 | EtOH/Water | 50 | 81 | XRD, IR | — |
| | Anti-solvent | | | | |
| | crystallization | | | | |
| 6 | CH₃CN | 80 | 75 | — | 163.9 |
| | Cooling | | | | |
| | crystallization | | | | |
| 7 | MIBK | 120 | 64 | XRD | 163.0 |
| | Cooling | | | | |
| | crystallization | | | | |
| 8 | 2-PrOH | 90 | 56 | XRD, IR | — |
| | Cooling | | | | |
| | crystallization | | | | |
| 9 | n-Butyl acetate | 135 | 83 | XRD | — |
| | Cooling | | | | |
| | crystallization | | | | |
| 10 | n-Propyl acetate | 110 | 62 | XRD | 163.6 |
| | Cooling | | | | |
| | crystallization | | | | |
| 11 | i-Butyl acetate | 130 | 71 | XRD | — |
| | Cooling | | | | |
| | crystallization | | | | |
| 12 | EtOH/water | 25 | 62 | XRD | — |
| | Neutralizing | | | | |
| | crystallization | | | | |
| 13 | EtOH/water | 10 | 87 | XRD, IR | 165.8 |
| | Suspension | | | | |
| 14 | EtOH/water | 50 | 89 | XRD, IR | 164.7 |
| | Suspension | | | | |
| 15 | EtOH/water | 25 | 53 | IR | — |
| | Neutralizing | | | | |
| | crystallization | | | | |
| 16 | EtOH/water | 0 | 32 | XRD | — |
| | Neutralizing | | | | |
| | crystallization | | | | |

The operation examples for representative examples will be described below. Other examples were also carried out according to the operation examples.

### Example 1

### Ethanol; Cooling Crystallization

To 1.54 g of (R)-3-[2-(2-amino-5-trifluoromethoxybenzamido)acetamido]-1-(6-methylindol-3-ylmethyl)pyrrolidine was added 3.55 mL of ethanol, followed by heating and dissolving in an oil bath at 70° C. The resultant solution was cooled to room temperature as is and then was settled overnight. The precipitated crystals were filtered off and dried. Yield Amount: 0.84 g (yield: 48%)

### Example 3

### Ethanol/Hexane; Cooling Crystallization

To 4.74 g of (R)-3-[2-(2-amino-5-trifluoromethoxybenzamido)acetamido]-1-(6-methylindol-3-ylmethyl)pyrrolidine was added 10 mL of ethanol, followed by heating and dissolving in an oil bath at 70° C. Then, the temperature of the oil bath was set at 80° C., followed by further adding 13 mL of ethanol, and subsequently adding 23 mL of hexane. The resultant solution was cooled to room temperature as is. The precipitated crystals were filtered off and dried. Yield Amount: 2.22 g (yield: 47%)

### Example 5

### Ethanol/Water; Anti-Solvent Crystallization

To 4.60 g of (R)-3-[2-(2-amino-5-trifluoromethoxybenzamido)acetamido]-1-(6-methylindol-3-ylmethyl)pyrrolidine was added 69 mL of ethanol, followed by dissolving in an oil bath while heating to 50° C. The resultant solution was cooled to room temperature as is, and then partly precipitated impurities were filtered out, followed by adding 69 mL of water to the filtrate. The precipitated crystals were filtered off and dried. Yield Amount: 3.72 g (yield: 81%)

### Example 13

### Ethanol/Water; Suspension

To 2.50 g of (R)-3-[2-(2-amino-5-trifluoromethoxybenzamido)acetamido]-1-(6-methylindol-3-ylmethyl)pyrrolidine was added 25 mL of ethanol, followed by adding 25 mL of water. The resultant clouded solution was stirred at temperature lower than 10° C. for 2 hrs, followed by filtering off the precipitated crystals and subsequently drying. Yield Amount: 2.22 g (yield: 89%)

### Example 16

### Ethanol/Water; Neutralizing Crystallization

Ten grams of (R)-3-[2-(2-amino-5-trifluoromethoxybenzamido)acetamido]-1-(6-methylindol-3-ylmethyl)pyrrolidine was dissolved in 40.8 mL of methanol and 122.6 mL of ethyl acetate. The resultant solution was washed with 61.3 mL of 0.5 M aqueous sodium hydroxide solution and 81.7 mL of 0.25 M aqueous sodium hydroxide solution. Incidentally, this operation is performed to remove impurities by extraction and washing, which may be omitted if not necessary.

Subsequently, 25.5 mL of 2M hydrochloric acid was added, followed by extracting the resultant (R)-3-[2-(2-amino-5-trifluoromethoxybenzamido)acetamido]-1-(6-methylindol-3-ylmethyl)pyrrolidine hydrochloride to the aqueous layer. Then 51.5 mL of ethanol was added and stirred under cooling on ice. To the solution was added 25.5 mL of 2 M aqueous sodium hydroxide solution. The precipitated crystals were filtered off and dried. Yield Amount: 3.19 g (yield: 32%)

### Example 17

### Production of Crystal B of (R)-3-[2-(2-amino-5-trifluoromethoxybenzamido)acetamido]-1-(6-methylindol-3-ylmethyl)pyrrolidine

(R)-3-[2-(2-amino-5-trifluoromethoxybenzamido)acetamido]-1-(6-methylindol-3-ylmethyl)pyrrolidine (16.39 g) was dissolved in a mixed solvent of 134 mL of ethanol and 50.3 mL of 2M hydrochloric acid. Meanwhile, a solution was prepared by adding 8.6 mL of water to 51.4 mL of 2 M aqueous sodium hydroxide solution. To the solution was added the acid solution prepared previously. The precipitated crystals were filtered off and dried. Yield Amount: 11.75 g (yield: 72%)

### Examples 18 to 23

### Comparison of Impurity Contents by a Crystallization Operation of (R)-3-[2-(2-amino-5-trifluoromethoxybenzamido)acetamido]-1-(6-methylindol-3-ylmethyl)pyrrolidine

The crystallization process operations shown in the examples in Table 4 were performed by using the initial sample (purity 97.44%, the content of major impurities 0.19% and 0.18%). The procedure was carried out according to the above-mentioned operation examples.

TABLE 4

| | | | | | Major |
|---|---|---|---|---|---|
| Exam- | Solvent | Tempera- | | | Impurities |
| ple | Crystallization | ture | Recovery | Purity | Contents |
| No. | process | (° C.) | Rate (%) | (%) | (%) |
| 18 | CH₃CN | 90 | 86.2 | 97.07 | 0.63 |
| | Cooling | | | | 0.41 |
| | crystallization | | | | |
| 19 | EtOH/water | 80 | 90.6 | 92.30 | 0.89 |
| | Cooling | | | | 5.48 |
| | crystallization | | | | |
| 20 | 2-PrOH | 90 | 83.8 | 96.02 | 1.34 |
| | Cooling | | | | 1.52 |
| | crystallization | | | | |
| 21 | n-Propyl acetate | 110 | 94.2 | 97.22 | 0.95 |
| | Cooling | | | | 0.53 |
| | crystallization | | | | |
| 22 | EtOH/water | 10 | 95.8 | 97.87 | 0.08 |
| | Suspension | | | | 0.17 |
| 23 | EtOH/water | 20 | 95.0 | 97.67 | 0.08 |
| | Neutralizing | | | | 0.01 |
| | crystallization | | | | |

As shown in Table 4, it has been clarified that the content of major impurities may be minimized, especially by the neutralizing crystallization.

### Example 24

### Production of Amorphous Form of (R)-3-[2-(2-amino-5-trifluoromethoxybenzamido)acetamido]-1-(6-methylindol-3-ylmethyl)pyrrolidine

Three hundred milligrams of (R)-3-[2-(2-amino-5-trifluoromethoxybenzamido)acetamido]-1-(6-methylindol-3-ylmethyl)pyrrolidine was dissolved in 5 mL of methanol, followed by dispersing in 150 ml, of water. (R)-3-[2-(2-amino-5-trifluoromethoxybenzamicdo)acetamido]-1-(6-methylindol-3-ylmethyl)pyrrolidine precipitated as an amorphous form was filtered off. Yield Amount: 164 mg (yield: 54.8%). It was confirmed by an XRD measurement that the precipitate was an amorphous substance.

### Example 25

### Transition of Crystal B of (R)-3-[2-(2-amino-5-trifluoromethoxybenzamido)acetamido]-1-(6-methylindol-3-ylmethyl)pyrrolidine into Crystal A

Five grams of (R)-3-[2-(2-amino-5-trifluoromethoxybenzamido)acetamido]-1-(6-methylindol-3-ylmethyl)pyrrolidine was suspended in 232 mL of ethanol/water (1.22/1), and the suspension was stirred for 3 hours in an oil bath at 20° C. The precipitated crystals were filtered off and dried at 50° C. under reduced pressure. Yield Amount: 4.25 g (yield: 85%)

HPLC pattern and XRD diagram were measured to confirm that the resulting crystal was crystal A of (R)-3-[2-(2-amino-5-trifluoromethoxybenzamido)acetamido]-1-(6-methylindol-3-ylmethyl)pyrrolidine.

This fact is disadvantageous in that conditions for drug products formation process that includes ordinary wet granulation may be restricted, because there is a risk of crystal form transition into crystal A as long as the process allows suspension status of crystal B in some solution during the process.

### Example 26

### Storage stability Comparison of Crystal A, Crystal B and Amorphous Form of (R)-3-[2-(2-amino-5-trifluoromethoxybenzamido)acetamido]-1-(6-methylindol-3-ylmethyl)pyrrolidine

Crystal A, crystal B and amorphous form of (R)-3-[2-(2-amino-5-trifluoromethoxybenzamido)acetamido]-1(6-methylindol-3-ylmethyl)pyrrolidine in the amount of 1.50 g, 1.50 g and 0.30 g, respectively were each placed in a transparent glass vial. They were simply covered by filter paper and kept in a thermostatic chamber. Samples were serially extracted for HPLC analysis and the appearance was also observed. Transition of crystal form was monitored by XRD analysis at the point of 163 hrs (only for crystals A and B) and 15 days (for all the samples) from the start. Purity of the drug substances was summarized in table 5. The “time” means storage time. The “temperature” is the preset temperature of the thermostatic chamber used for the storage, which was jumped up at 163 hrs from the start.

TABLE 5

| | | Temper- | | | Purity of |
|---|---|---|---|---|---|
| | | ature | Purity of | Purity of | amorphous |
| Time | | (° C.) | Crystal A (%) | Crystal B (%) | form (%) |
| Initial | | 40 | 98.79 | 99.20 | 98.03 |
| value | | | | | |
| 24 | hrs | 40 | 98.89 | 99.22 | 97.78 |
| 71 | hrs | 40 | 98.88 | 99.31 | 97.55 |
| 144 | hrs | 40 | 98.80 | 99.22 | 97.25 |
| 163 | hrs | 40 to 60 | Crystal A | Crystal B | — |
| | | | (XRD) | (XRD) | |
| 192 | hrs | 60 | 98.82 | 99.28 | 95.88 |
| 240 | hrs | 60 | 98.82 | 99.19 | 94.33 |
| 15 | days | 60 | 98.75 | 99.28 | 91.14 |
| | | | Crystal A | Crystal B | amorphous |
| | | | (XRD) | (XRD) | form (XRD) |

As shown in table 5, degradation of amorphous form of (R)-3-[2-(2-amino-5-trifluoromethoxybenzamido)acetamido]-1-(6-methylindol-3-ylmethyl)pyrrolidine was observed even at 40° C. This means that amorphous form of the compound has less storage stability compared to the crystal forms of the compound. Therefore, the crystal forms are more preferable for pharmaceutical use in this regard.

### INDUSTRIAL APPLICABILITY

A crystal or an amorphous form of (R)-3-[2-(2-amino-5-trifluoromethoxybenzamido)acetamido]-1-(6-methylindol-3-ylmethyl)pyrrolidine is used for production of a pharmaceutical product.

1. A crystal of (R)-3-[2-(2-amino-5-trifluoromethoxybenzamido)acetamido]-1-(6-methylindol-3-ylmethyl)pyrrolidine.

2. A crystal of (R)-3-[2-(2-amino-5-trifluoromethoxybenzamido)acetamido]-1-(6-methylindol-3-ylmethyl)pyrrolidine (crystal A) exhibiting an X-ray powder diffraction pattern having characteristic peaks expressed in the reflection angle 2θ (degree) at about 5.7, 8.4, 15.2, 16.9, 19.7, 20.9, 21.3, 21.7 and 24.1.

3. A crystal of (R)-3-[2-(2-amino-5-trifluoromethoxybenzamido)acetamido]-1-(6-methylindol-3-ylmethyl)pyrrolidine (crystal A) exhibiting an X-ray powder diffraction pattern approximately shown in FIG. 1.

4. A crystal of (R)-3-[2-(2-amino-5-trifluoromethoxybenzamido)acetamido]-1-(6-methylindol-3-ylmethyl)pyrrolidine (crystal B) exhibiting an X-ray powder diffraction pattern having characteristic peaks expressed in the reflection angle 2θ (degree) at about 9.6, 11.3, 15.5, 16.3, 16.9, 19.3, 20.0, 20.5, 20.9, 22.7, 23.3, 24.2, 27.2, 27.8 and 31.6.

5. A crystal of (R)-3-[2-(2-amino-5-trifluoromethoxybenzamido)acetamido]-1-(6-methylindol-3-ylmethyl)pyrrolidine (crystal B) exhibiting an X-ray powder diffraction pattern approximately shown in FIG. 2.

6. A crystal of (R)-3-[2-(2-amino-5-trifluoromethoxybenzamido)acetamido]-1-(6-methylindol-3-ylmethyl)pyrrolidine (crystal A) exhibiting an infrared absorption spectrum in potassium bromide having absorption peaks expressed in the wavenumber (cm⁻¹) at approximately 1651, 1637, 1583, 1556, 1294, 1265, 1223, 1205, 1169, 1155, 1097, 1051, 1007, 966, 885, 835 and 804.

7. A crystal of (R)-3-[2-(2-amino-5-trifluoromethoxybenzamido)acetamido]-1-(6-methylindol-3-ylmethyl)pyrrolidine (crystal A) exhibiting an infrared absorption spectrum in potassium bromide having the absorption pattern approximately shown in FIG. 3.

8. A crystal of (R)-3-[2-(2-amino-5-trifluoromethoxybenzamido)acetamido]-1-(6-methylindol-3-ylmethyl)pyrrolidine (crystal B) exhibiting an infrared absorption spectrum in potassium bromide having absorption peaks expressed in the wavenumber (cm⁻¹) at approximately 1639, 1556, 1265, 1223, 1167, 1149, 1119, 1099, 1055, 1011, 960, 891, 858, 825 and 796.

9. A crystal of (R)-3-[2-(2-amino-5-trifluoromethoxybenzamido)acetamido]-1-(6-methylindol-3-ylmethyl)pyrrolidine (crystal B) exhibiting an infrared absorption spectrum in potassium bromide having the absorption pattern approximately shown in FIG. 4.

10. An amorphous form of (R)-3-[2-(2-amino-5-trifluoromethoxybenzamido)acetamido]-1-(6-methylindol-3-ylmethyl)pyrrolidine.

11. A method of producing crystal A by cooling crystallization of a solution of (R)-3-[2-(2-amino-5-trifluoromethoxybenzamido)acetamido]-1-(6-methylindol-3-ylmethyl)pyrrolidine in methanol, ethanol, 2-propanol, ethyl acetate, n-propyl acetate, tetrahydrofuran, 2-butanone, acetonitrile, toluene, hexane, heptane, water or a mixed solvent of two kinds or more selected thereof.

12. A method of producing crystal A by anti-solvent crystallization process, wherein toluene, hexane, heptane, water or a mixed solvent of two kinds or more selected thereof is added to a solution of (R)-3-[2-(2-amino-5-trifluoromethoxybenzamido)acetamido]-1-(6-methylindol-3-ylmethyl)pyrrolidine in methanol, ethanol, 2-propanol, ethyl acetate, n-propyl acetate, tetrahydrofuran, 2-butanone, acetonitrile, toluene, hexane, heptane, water or a mixed solvent of two kinds or more selected thereof.

13. A method of producing crystal A by carrying out a cooling crystallization step from a solution of (R)-3-[2-(2-amino-5-trifluoromethoxybenzamido)acetamido]-1-(6-methylindol-3-ylmethyl)pyrrolidine in methanol, ethanol, 2-propanol, ethyl acetate, n-propyl acetate, tetrahydrofuran, 2-butanone, acetonitrile, toluene, hexane, heptane, water or in a mixed solvent of two kinds or more selected thereof, simultaneously with or before an anti-solvent crystallization step, wherein toluene, hexane, heptane, water or a mixed solvent of two kinds or more selected thereof is added further.

14. A method of producing crystal A by suspending (R)-3-[2-(2-amino-5-trifluoromethoxybenzamido)acetamido]-1-(6-methylindol-3-ylmethyl)pyrrolidine in methanol, ethanol, 2-propanol, ethyl acetate, n-propyl acetate, tetrahydrofuran, 2-butanone, acetonitrile, toluene, hexane, heptane, water or a mixed solvent of two kinds or more selected thereof.

15. A method of producing crystal A by neutralizing crystallization by adding an alkaline solution or a water soluble organic solvent containing the alkaline solution to a solution of an acid salt or a mixture of acid salts of (R)-3-[2-(2-amino-5-trifluoromethoxybenzamido)acetamido]-1-(6-methylindol-3-ylmethyl)pyrrolidine in water or in a mixed solvent of water and two or more solvents selected from water soluble organic solvents.

16. A method of producing crystal B by neutralizing crystallization by adding a solution of acid salt of (R)-3-[2-(2-amino-5-trifluoromethoxybenzamido)acetamido]-1-(6-methylindol-3-ylmethyl)pyrrolidine in water or in a mixed solvent of water and two or more solvents selected from water soluble organic solvents to an alkaline solution or a water soluble organic solvent containing the alkaline solution.

17. A pharmaceutical composition containing, as an active ingredient, a crystal or an amorphous form, or a mixture of the crystal or the amorphous form selected therefrom according to claim 1.

18. A chemokine receptor antagonist composition containing, as an active ingredient, a crystal or an amorphous form, or a mixture of the crystal or amorphous form selected therefrom according to claim 1.

19. A preventive drug or a therapeutic drug for inflammatory disease, allergic disease, respiratory disease or cardiovascular disease containing, as an active ingredient, a crystal form or an amorphous form, or a mixture of the crystal or amorphous form selected therefrom according to claim 1.
